(19) United States
(12) Patent Application Publication
(10) Pub. No.: US 2020/0008395 A1
(43) Pub. Date: Jan. 9, 2020

(54) METHOD OF USING HUMAN EXCREMENT AND SECRETIONS FOR EARLY DETECTION OF PARKINSON'S DISEASE

(71) Applicant: NEURRX, SPC, Shoreline, WA (US)

(72) Inventor: Laurie Kathern MISCHLEY, Shoreline, WA (US)

(21) Appl. No.: 16/512,301

(22) Filed: Jul. 15, 2019

Related U.S. Application Data
(63) Continuation-in-part of application No. PCT/US2018/013633, filed on Jan. 12, 2018.
(60) Provisional application No. 62/446,433, filed on Jan. 14, 2017.

(51) Int. Cl. A01K 15/02; G01N 33/48

(52) U.S. Cl. CPC A01K 15/02; G01N 2800/2835; G01N 33/48

(57) ABSTRACT

Provided herein are methods for early detection of Parkinson's diseases (PD), especially in individuals showing no motor symptoms, by detecting PD-distinct scent using a trained animal (such as a canine). Specific rigorous animal training in scent identification through both patient contact and training samples are also disclosed.

### CROSS-REFERENCE TO RELATED APPLICATIONS

This application is a continuation-in-part application of International Application No. PCT/US2018/013633, filed Jan. 12, 2018, which claims the benefit of the filing date of U.S. Provisional Application No. 62/446,433, filed Jan. 14, 2017, which applications are incorporated herein by reference in their entireties.

### BACKGROUND

### Technical Field

The present invention relates to the field of clinical markers and early detection methodology for idiopathic Parkinson's disease and related parkinsonian syndromes.

### Description of the Related Art

At present, Parkinson's disease (PD) is defined pathologically by the accumulation of Lewy bodies in the substantia nigra and thus, the disease cannot be conclusively diagnosed until autopsy.

Clinically, the diagnosis is made based on the presence of cardinal motor symptoms, such as tremor, rigidity, impaired balance, and stooped posture. Unilateral onset and clinical response to dopaminergic medications could aid in the clinical diagnosis. In some instances, providers may use an ioflupane I (¹²³I) injection to aid in the diagnosis. Also known as DaTScan™, ioflupane is an FDA-approved visual adjunct imaging agent indicated for striata dopamine transporter visualization using single-photon emission computed tomography (SPECT) brain imaging. Like other aids in clinical diagnosis of PD, this costly test does not confirm the disease.

As the understanding of the disease evolves, diagnostic criteria can be expected to change over time. It is generally recognized, however, that once the motor symptoms are detected, the disease progression is at a late stage and irreversible. Therefore, there is a particularly pressing need for early detection, preferably before the onset of the motor symptoms. Early detection provides the opportunity for personal and public health planning and preparedness, as well as implementation of disease modifying strategies as a preventive measure against disease progression.

### BRIEF SUMMARY

The present disclosure provides methods for detection of PD (including all parkinsonian disorders, as defined herein) in human test subjects, especially for early detection in test subjects showing no clinical symptoms (i.e., prodromal PD or pre-motor PD), by employing animals trained to detect volatile compounds in scent emitted from the body or bodily excrement of the test subjects. In particular, according to various embodiments described herein, animals having superior olfactory capacity such as canine and swine can be trained to detect. with reliability and repeatability, the unique and distinctive scents from people with parkinsonism (PwP) and signal such detection to a human handler. Advantageously, these trained animals can be employed to detect in a test subject the presence of Parkinson-distinctive scents even before the test subject shows any motor or other clinical symptoms.

Correct identification of Parkinson-distinctive scent requires positive reinforcement of the desired behavior (i.e., detection and signaling) during a multi-stage training process, in which an animal, typically a canine, begins the training with a positive association of the Parkinson-distinctive scent with a reward (e.g., a treat); followed by training the animal to discriminate between Parkinson-distinctive scent from other scents (controls). Correct identification can take any form so long as the animal's response to the Parkinson-distinctive scent can be signaled to the human handler in a consistent and reliable manner. For example, the animal may bark or wag its tail at a sample having Parkinson-distinctive scent or lead the handler to the sample.

Thus, one embodiment provides a method for training an animal for detecting and recognizing Parkinson's disease-distinctive scent, the method comprising:

providing a training sample including excrements derived from one or more Parkinson's disease (PD) sources, each PD source being a person having shown at least one clinical marker for PD;

providing a control sample including excrements derived from one or more persons showing no clinical marker for PD;

allowing the animal to associate the training sample with PD by exposing the animal to a training sample in combination with a treat;

training the animal to discriminate between the training sample and the control sample by allowing the animal to smell the training sample and the control sample in the absence of any treat and rewarding the animal with a treat only when the animal correctly identifies the training sample.

Another embodiment provides a training sample having Parkinson-distinctive scent, wherein the training sample comprises one or more types of excrements derived from one or more PD sources, each PD source being a person having shown at least one clinical marker for PD. The types of excrement include, without limitation, ear wax, skin cells (including sebum, sweat, epithelial cells, and dermal microbiota and/or the metabolites from the microbial organisms); stool, urine, saliva, and exhaled breath. The sample may be concentrated (e.g., from urine, saliva or sweat) or in a naturally extracted form (e.g., ear wax, skin cells, or stool).

A further embodiment provides a control sample having one or more types of excrement derived from one or more non-PD sources, i.e., persons having shown no clinical marker for PD. The control sample provides a base line scent against which an animal in training or a trained animal can discriminate when identifying a sample having Parkinson-distinctive scent. The control sample is important for training and sharpening the sensitivity of the animal's detecting capability. Thus, the control sample is also derived from the same types of excrements as those in the training sample, except that the sources of the excrements are persons showing no clinical markers for PD. Preferably, the sources are persons who have not shown any pre-motor symptoms either, such as loss of smell, constipation, restless leg syndrome, depression, rapid eye movement sleep behavior disorder (RBD) and the like.

As used herein, a clinical marker for PD (or related forms of parkinsonism) may be any marker that is presently recognized or may be developed in the future. In non-limiting examples, clinical markers may be levodopa-responsiveness, bradykinesia (slowness of initiation of voluntary movement with progressive reduction in speed and amplitude of repetitive actions), unilateral onset (symptoms started on one side of the body), progressive disorder (the disease has gotten worse over time), muscular rigidity, resting tremor, postural instability (not caused by primary visual, vestibular, cerebellar, or proprioceptive dysfunction), cognitive impairment and the like.

A further embodiment provides a method for detecting PD in a test subject, the method comprising: allowing a trained animal that has been trained to identify Parkinson-distinctive scent to smell the test subject or a test sample containing excrement extracted from the test subject; and interpreting a response given by the trained animal to determine the presence or absence of Parkinson-distinctive scent.

In order to improve confidence in the detection, the trained animal is typically provided a sample track that includes one or more test samples (e.g., duplicates or triplicates), one or more negative control samples derived from non-PD sources, and one or more positive or PD standard samples derived from one or more PD sources. Statistical analysis may be performed to evaluate the confidence in the responses by the animal.

A further embodiment provides a method of preventing or delaying the onset of motor and other clinical symptoms of PD in a test subject tested positive for Parkinson-distinctive scent by a trained animal, the method comprising subjecting the test subject to one or more PD therapies or modifications.

### BRIEF DESCRIPTION OF THE SEVERAL VIEWS OF THE DRAWINGS

FIG. 1 is a visual demonstration of how early treatment slowing PD is increasingly valuable the earlier the treatment is initiated.

FIG. 2 shows the PRO-PD rating scale, which could be used to screen and identify individuals with non-motor/nonspecific symptoms of PD, thereby increasing the likelihood of early detection.

FIG. 3 is a flowchart which shows, according to an embodiment, the order and stages of training a canine to detect and identify Parkinson-distinctive scent.

FIG. 4 shows a sample collection device, which houses a cotton swab used to extract excrement (e.g., ear wax) from a test subject or a PD source.

FIG. 5 shows a sample track according to one embodiment that contains a test sample, a control sample derived from non-PD sources and a standard sample derived from PD sources.

FIG. 6 shows an example of an animal trained according to the methodology described here, which in turn detected test samples with a positive predictive value of 78.95% (95% CI 61.83, 89.67%) and a negative predictive value of 100% in its ability to distinguish between samples from PD sources vs. control samples from non-PD sources.

FIG. 7 shows an exemplary process of screening individuals in a general population to identify those at the greatest risk of parkinsonism and implementing intervention strategies for individuals that tested positive to delay or prevent the onset of parkinsonism.

### DETAILED DESCRIPTION

Disclosed herein are methods for detecting Parkinsonian disorders (PD) in human subjects by the detection of Parkinson-distinctive scent. In particular, animals can be trained to identify Parkinson-distinctive scent in a human subject during the prodromal phase, before the subject even develops motor or other clinical symptoms. Early detection and intervention can be effective in preventing or delaying the onset of PD symptoms.

### Parkinsonian Disorders and the Value of Early Detection

Parkinsonian disorders (also referred to as Parkinsonism) may take different forms. The histological changes that have historically differentiated idiopathic Parkinson's disease (iPD) from other forms of parkinsonism are now understood to occur a decade or more into the disease and therefore not useful for prevention efforts. Clinically and cellularly, iPD, Alzheimer's disease (AD), Lewy body dementia (LBD), multiple system atrophy (MSA), corticobasal degeneration (CBD), progressive supranuclear palsy (PSP), etc. have much in common. There is emerging evidence that there may be more overlap between these disorders than was once thought, that many of these seemingly clinically distinct diagnoses are manifestations arising from a singular upstream insult. As used herein, PD refers to the entirety of the general spectrum of parkinsonian disorders, whereas iPD refers only to those with the more specific clinical diagnosis, e.g. meeting UK Brain Bank Criteria.

Thus, Parkinsonian disorders and Parkinson's disease are used interchangeably with terms such as parkinsonism, Parkinson-plus syndromes, Lewy Body dementia, Alzheimer's disease, and the related spectrum of neurodegenerative disorders (collectively referred to as PD). These terms have quasi-useful clinical distinctions with notable overlap. There is currently no reason to believe that each has a distinct pathophysiological process upstream. Clinical symptoms do not necessarily correlate with evidence of PD on imaging or autopsy; individuals with idiopathic PD are often told a few years into their disease that they have other forms of parkinsonism. Methods disclosed herein are aimed at addressing this diagnostic uncertainty and providing clarity to PD diagnostics.

Given the considerable overlap between various forms of PD, clinical distinction between them is of questionable relevance. There is emerging evidence, however, that PD is less of a disease and more of a collection of syndromes. All objective motor symptoms occur late in the disease and there is little to no evidence the modification of motor aspects of the disease slows the underlying disease course. These motor symptoms include, for example, bradykinesia (slowness of initiation of voluntary movement with progressive reduction in speed and amplitude of repetitive actions), unilateral onset (symptoms started on one side of the body), progressive disorder (the disease has gotten worse over time, muscular rigidity, 4-6 Hz resting tremor, postural instability (not caused by primary visual, vestibular, cerebellar, or proprioceptive dysfunction).

Clinical diagnosis of PD based on motor symptoms occurs late in the disease. However, there is evidence that some cohorts may have constipation up to 18 years prior to the onset of PD motor symptoms. There is also evidence of protein aggregation and Lewy bodies in the intestinal mucosa and salivary glands of individuals with PD approximately a decade prior to onset of motor symptoms. Thus, it has been recognized that PD presents pre-motor symptoms such as loss of smell, constipation (defined by less than one bowel movement per day), restless leg syndrome, depression, and symptoms associated with rapid eye movement sleep behavior disorder (RBD), e.g., “acting out their dreams” while asleep, such as punching, flailing arms in the air, making running movements, etc. These pre-motor symptoms in themselves are not necessarily predictive of PD, but are often experienced by PD patient prior to receiving their clinical diagnosis. When experienced by individuals who ultimately receive a clinical diagnosis of PD, such pre-motor symptoms are also referred to herein as pre-motor PD.

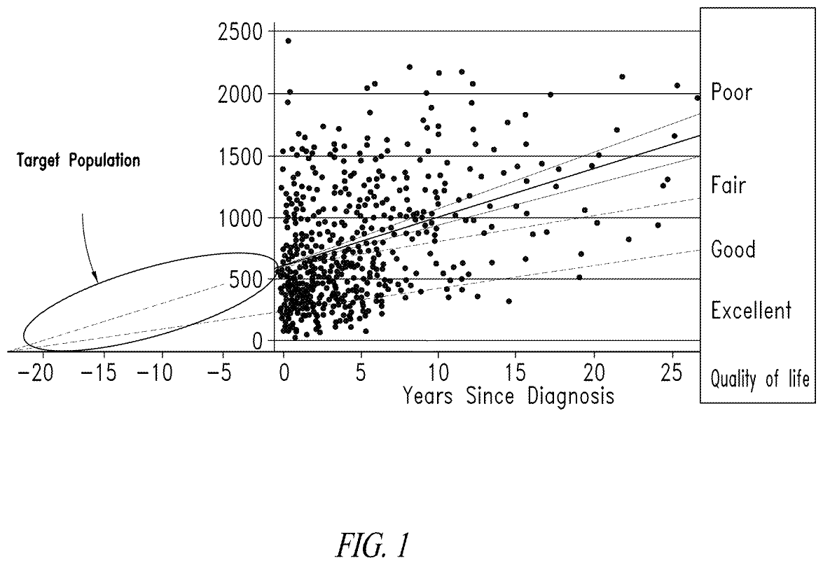

When the diagnosis comes 10-15 years or more into the disease, the contribution or effectiveness of a disease-modifying therapy is diminished. Conversely, early detection and intervention by disease-modifying therapies can reduce rate of PD progression or delay disease onset. FIG. 1 demonstrates visually interventions associated with reduced rate of PD progression. For instance, implementation of an intervention in the individual with pre-motor PD can reduce the mean rate of PD progression from 37 points/year to <16 on the PRO-PD scale. FIG. 1 further shows that the reduction in rate (i.e., slope) of disease progression means less when the treatment is initiated later in the course of the disease. As used herein, PRO-PD is the Patient Reported Outcomes in Parkinson's Disease rating scale, which is used to describe PD disease severity among individuals with a clinical diagnosis of PD.

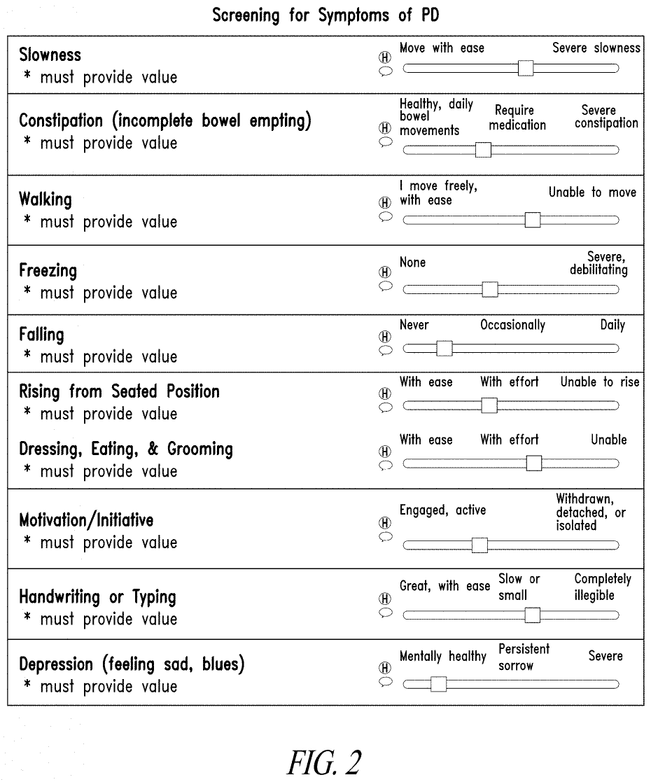

FIG. 2 shows the PRO-PD rating scale, which could be used to screen and identify individuals with non-motor/nonspecific symptoms of PD, thereby increasing the likelihood of early detection.

Early detection accompanied by disease modification is of tremendous value to the affected individual and the health care system. Disease modifications such as targeted exercise interventions, nutritional supplements, food choices, and social health are associated with rate of PD progression. Thus, early identification of pre-motor PD is capable of identifying individuals most likely to benefit from interventions that slow, stop, and prevent progression of parkinsonism.

Even without employing disease-modification strategies, the knowledge of potential onset of PD may improve personal and public health planning and preparedness.

### Parkinson-Distinctive Scent

People with PD, including pre-motor and prodromal PD, have “unique molecular profiles,” which are likely to overlap with clinical symptoms. These “unique molecular profiles” are associated with an “aromatic cloud,” also referred to as Parkinson-distinctive scent. At least in one incident, the distinct scent in PD was reportedly detectable by a human who, after having attended to her husband inflicted with PD, could identify PD sufferers by their smells. See Kwon D, One Woman's Ability to Sniff Out Parkinson's Offers Hope to Sufferers: A musky odor may lead to new diagnostic tools for the neurodegenerative disease, https://www.scientificamerican.com/article/one-woman-s-ability-to-sniff-out-parkinson-s-offers-hope-to-sufferers/, Accessed Jan. 3, 2017.

The method described in the present disclosure is particularly advantageous because it is capable of detecting Parkinson-distinct scent before an individual even exhibits motor symptoms or receives a PD clinical diagnosis (i.e., pre-motor/prodromal PD) and because it enables samples from individuals to be sent to a lab for detection. As discussed herein, such early detection enables an individual to seek disease-modifying therapy at a time when the individual can most benefit from interventions that prevent or delay PD progression.

The detection is not specific to a particular form of parkinsonism, such as idiopathic Parkinson's disease (iPD). While it may be possible in the future to train animals to discern between pathologically and phenotypically different types of parkinsonism, presently the animals are unable to distinguish between iPD and Parkinson-plus syndromes such as multiple system atrophy, Kufor Rakeb, Lewy body dementia, Alzheimer's disease, etc. Rather, the animals are trained to detect the distinctive scent from a human subject who is either symptomatic for PD or prodromal PD, and can distinguish it from scents from a non-PD source.

### Animals Trained to Detect Parkinson-Distinctive Scent

The animals used herein are non-human species selected for their superior sense of smell compared to that of humans'. Canines and swine are particularly preferred because they are domesticated and have been proven to be trainable to detect substances such as explosives and contrabands. Both dogs and pigs have been bred for hunting truffles, which grow underground and produce a distinct odor when ripe. More recently, dogs have been utilized in clinical diagnosis of cancer and other medical indications requiring odorant profiles, e.g., blood sugar in diabetics. Other animals such as boars, goats, ferrets, coyotes, foxes, raccoons and the like also possess superior olfactory capability and may be trained according to the methods disclosed herein.

In preferred embodiments, the animal is a canine. In a particular example, the animal is a breed of dog called Lagotto Romagnolo, which has been bred for its ability to detect truffles. The breed is known for their intelligence, sense of smell, calm nature, and capacity to communicate with their human handler/trainer when the target has been found. While the animals or canines used for the purposes of identifying Parkinson-distinctive scent need not be a Lagotto Romagnolo, this canine breed exemplifies the characteristics important for success.

### Training Animals

One embodiment provides a method for training an animal for detecting and recognizing Parkinson's disease-distinctive scent, the method comprising: providing a training sample including excrements derived from one or more Parkinson's disease (PD) sources, each PD source being a person having shown at least one clinical marker for PD; providing a control sample including excrements derived from one or more persons showing no clinical marker for PD; allowing the animal to associate the training sample with PD by exposing the animal to a training sample in combination with a treat; training the animal to discriminate between the training sample and the control sample by allowing the animal to smell the training sample and the control sample in the absence of any treat and rewarding the animal with a treat only when the animal correctly identifies the training sample.

1. Parkinson-Scented Samples

For purposes of early training and orienting the dog to the Parkinson-distinctive scent on a regular basis, an animal is initially exposed to Parkinson-distinctive scent and associates it with a reward (e.g., a treat). All humans have excretions and secretions (collectively referred to as “excrements”) that contain aromatic remnants of an individual's metabolic profile. Parkinson-scented samples may be collected from individuals that have an established diagnosis of Parkinson's disease (also referred to as PD source). The Parkinson-scented samples may be used as a training sample for training or as a positive control (i.e., a PD standard sample) for detecting.

Biological samples or excrements may be collected from the ear canal, anus, saliva, throat, skin, and urine of one or more PD sources. Thus, the types of excrements that may be used in the training sample include, for example, cerumen (ear wax), skin cells (e.g., taken from underarms, face, feet, groin, which include a combination of sebum, sweat, epithelial cells, and dermal microbiota), organisms and their metabolites from the dermal microbiome, stool (including intestinal microbiome, sloughed intestinal epithelial cells, byproducts of metabolism), urine, sebum, saliva, and exhaled breath.

In some embodiments, biological samples may be collected directly with a swab, pad, wipe, tissue, or other physical carrier or collectors. For example, a cotton-tipped swab may be inserted into the ear to retrieve sebum, skin cells, and ear wax. A wipe (optionally soaked with alcohol or other solvents) may be used to collect excrements such as sebum from skin.

When used as a training sample, the biological samples can be placed in an air-tight container. The container has one perforated side to allow the aromatic profile of the individual(s) to escape during detection, and a lid that can be applied to prevent the aromatic cloud from escaping when not in use.

In preferred embodiments, various excrements are collected from multiple PD sources and combined into one sample. The combination increases the chances that the Parkinson-distinctive scent is comprehensively captured. In one embodiment, the training sample is a blend of excrements of the same type (e.g., all ear wax) from different PD sources. There is no limit on the number of the PD sources from whom the excrements may be collected and combined. Samples from five distinct individuals were combined for the PD training samples used to gather the data presented herein.

Thus, in various embodiments, biological samples or excrements may be collected from one or more individuals who have shown at least one clinical marker for PD. For example, the individual has reported levodopa-responsiveness. Exemplary levodopa responsiveness include, for example, they have used carbidopa/levodopa for at least 30 days (e.g., at a dose of at least 25 mg carbdopa/100 mg levodopa 3×/day); their PD symptoms have improved by 70-100% from using levodopa, or they have had a sustained response to levodopa for 5 years or more.

Biological samples or excrements may also be extracted from individuals who have exhibited other clinical markers such as one or more motor symptoms. Non-limiting examples include bradykinesia (slowness of initiation of voluntary movement with progressive reduction in speed and amplitude of repetitive actions); unilateral onset (symptoms started on one side of the body); progressive disorder (the disease has gotten worse over time); muscular rigidity; 4-6 Hz resting tremor or postural instability (not caused by primary visual, vestibular, cerebellar, or proprioceptive dysfunction).

2. Control Samples from Non-PD Sources

Control samples from healthy, non-PD sources are needed for training animals to distinguish Parkinson-distinctive scent from non-Parkinson scents and for using as a negative control in detection (“Control Standard”). Biological samples or excrements are collected from individuals who are not PD sources, namely, they are not given a PD diagnosis. Preferably, the non-PD sources are also individuals who have not shown symptoms that could potentially indicate pre-motor or non-motor PD. Thus, non-PD sources suitable for providing control samples are individuals who have not received PD diagnoses or exhibited any of the following potential pre-motor PD symptoms: loss of smell; constipation; restless leg syndrome; depression; rapid eye movement sleep behavior disorder (RBD), fatigue, impaired sleep, impaired balance, or a combination thereof.

Identical specimen collection procedures are followed for both individuals with established PD and those considered non-PD controls.

3. Training the Animals

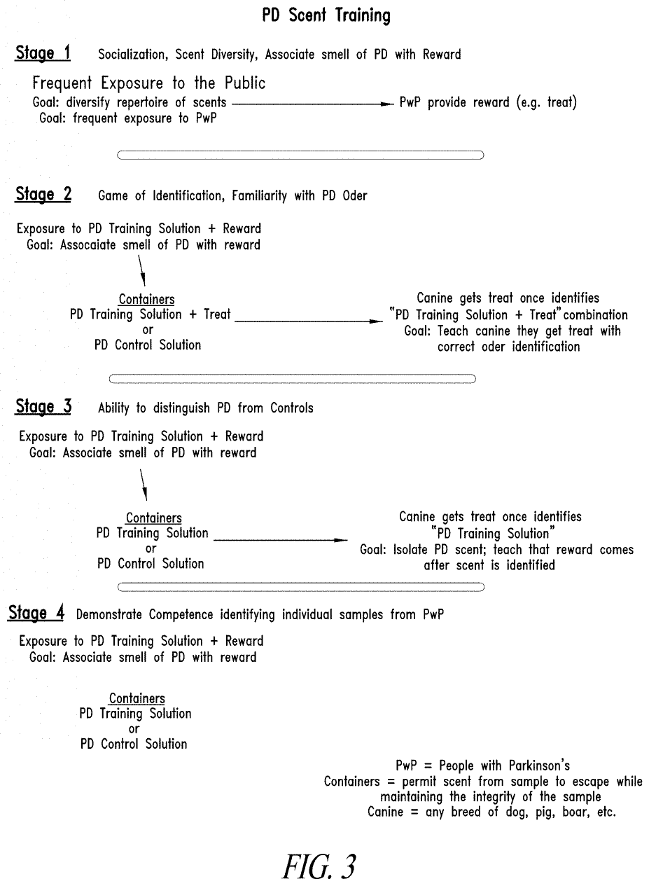

FIG. 3 shows a flowchart of training the animals to identify and respond to Parkinson-distinctive scent. Various positive-reinforcement techniques can be adopted at all stages of training. For establishing a positive association or connection to the Parkinson-distinctive scent, the animals are rewarded with treats or emotions/affections from the human handler, who may congratulate them, show pleasure, play with their favorite toy, etc.

Animals should be started at the youngest age possible in scent training. Optionally, they are exposed to the public as well as to the PD patients. Frequent exposure allows the animals to accumulate a diverse repertoire of scents. When the animals are exposed to the PD patients (PwP), they are given treats to associate the Parkinson-distinctive scent to PD. Although not required, the animal benefits from frequent clinical exposure to individuals with parkinsonism. Examples of clinical exposure may be for the handler that works with individuals with parkinsonism (e.g., physician, nurse, physical therapist, etc.) to bring the animal to work. The handler orients the animal to the Parkinson-distinctive scent at the start of the day and provides a reward each time the animal correctly signals an established patient. Another example is to bring the animal to Parkinson's related events, where it is exposed to high concentrations of individuals with PD.

Other training “games” with built-in positive reinforcements include: exposing the animal to a Parkinson-scented training sample followed by a reward; placing the Parkinson-scented training sample under one of two or three identical upside-down bowls, moving the bowls around, mixing them up, and rewarding the animal for identifying the bowl that has the PD scent underneath; playing hide-and-seek in an environment (e.g., home) where the Parkinson-scented training sample is hidden in increasingly more difficult locations and rewarding the animal for finding and alerting to the scent; participating in scent-identification activities other than the Parkinson-distinctive cent, e.g., truffle hunting, search and rescue, etc. The animals are also trained to distinguish or discriminate between

Parkinson-distinctive scent and non-Parkinson scent from a control sample by exposing the animals to both samples and only rewarding the animals when the Parkinson-distinctive scent has been identified.

The animals are not only trained to identify the aromatic profiles of the samples, but will be trained to clearly and consistently signal to the handler when given the command learned in training. It is not anticipated that all animals will give the handler the same signal, although it is expected that the signal should be identifiable to a lay observer.

4. Training of the Handler and Optimization of the Training Environment

The human handler will be trained in the science of reward and basic principles of scent work. Preferably, the handlers are also familiar with other successful uses of canine scent work, including, for example, detection of explosives; detection of illicit substances; medical detection (e.g., predicting seizures, hypoglycemia, cancer, etc.); search and rescue for missing persons.

It is important to optimize the training environment, which includes both work and home where the animals are present. For example, home and work environment must be free of scented perfumes, lotions, etc. Handler should be made familiar with established risks to olfactory health.

### Identifying Individuals at Increased Risk of Developing Motor Symptoms of Parkinson's Disease

A trained animal can be employed to identify individuals who are pre-motor PD but at an increased risk of developing motor symptoms of PD. The individual (i.e., a test subject) may be screened (i.e., smelled) directly by a trained animal or provide a biological test sample for the animal to screen.

1. Pre-screening Test Subjects

While a biological sample from any individual can be tested, the sensitivity and specificity of the test are expected to increase as the individual becomes more phenotypically similar to individuals with parkinsonism.

Thus, the test subjects may be pre-screened to improve diagnostic accuracy according to the following non-limiting criteria: (1) testing all individuals above a certain age (e.g., 40 and up) because parkinsonism increases with age; (2) identifying a specified number (e.g., at least two or three) of symptoms common to PD: loss of smell, irritable bowel syndrome/constipation, handwriting impairment, impaired sleep, fatigue, muscle pain, forgetfulness, sexual impairment, and mood disorders; (3) testing individuals who score above a certain level on the Patient-Reported Outcomes in PD rating scale, for instance, level to be mathematically determined with increasing availability of data, e.g., 300-500 (see also FIG. 2); (4) testing any individual a physician identifies as having a possible symptom of PD, e.g., masked face, lack of arm swing, etc.; and (5) periodic testing of individuals who carry a gene that increases their risk of developing PD. Genes known to be associated with increased risk of PD include, for example, PARK1; alpha-synuclein (SNCA); leucine-rich repeat kinase 2 (LRRK2); PTEN-induced putative kinase I (PINK1); glucocerebrosidase (GBA).

In some embodiments, the non-motor symptom(s) may be a collection of one or more of the following symptoms:

(a) abnormal gastrointestinal symptoms, e.g., constipation, diarrhea, bloating;

(b) impaired handwriting and/or difficulty typing.

(c) fatigue.

(d) disrupted sleep, e.g., insomnia, nighttime waking, REM sleep behavior disorder.

(e) mood disorders, e.g., anxiety, depression, dysthymia, apathy.

hyposmia (loss of ability to smell).

(g) sexual impairment, e.g., erectile dysfunction, inability to orgasm.

(h) urinary dysfunction, e.g., urgency, incontinence

Detection by the trained animal according to the method disclosed herein, accompanied by the above pre-screening methodologies, can increase the sensitivity and accuracy of PD diagnosis.

2. Test Samples

Any individual and/or physician who wants to rule out a form of parkinsonism may submit a test sample. Individuals will be given instructions related to, and materials for, sample collection, storage, and transportation, as appropriate for the kit. The kit will be designed to capture the aromatic profile of the individual and minimize the need for handling upon receipt at the lab. At the facility, trained animals will be used to determine whether the test is positive or negative.

Biological test samples may include scrapings from ear canal, anus, saliva, throat, skin, and urine. The ultimate combination of biological excrements used will depend on the sensitivity and specificity of individual samples and/or combinations, with respect to the individual's ability and willingness to collect and transport the sample. For instance, a fecal sample may yield a 3% improvement in specificity over earwax, but result in a 40% reduction in compliance with sample submission.

In some embodiments, test samples may be collected directly with a swab, pad, tissue, wipe or other physical carrier or collectors. For example, a cotton-tipped swab may be inserted into the ear to retrieve sebum, skin cells, and ear wax. An alcohol-soaked wipe may be used to collect sebum from skin (e.g., taken from underarms, face, feet, groin, etc.)

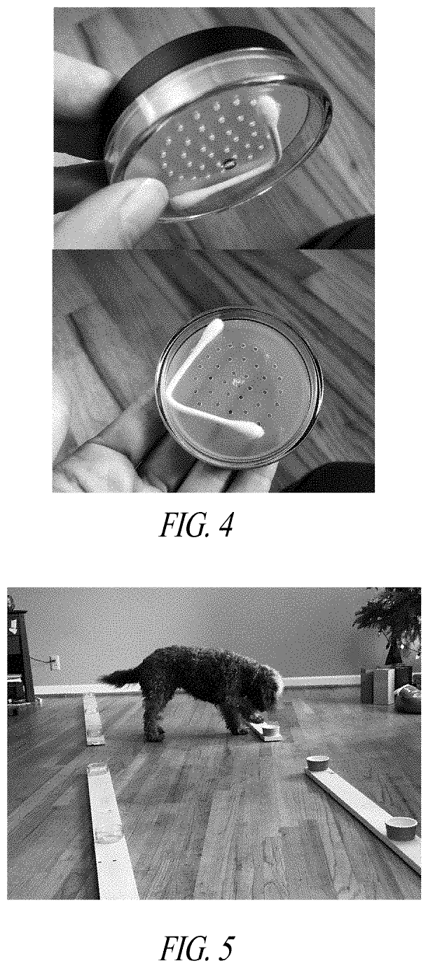

Test samples are collected by the individual test subject and placed in an air-tight container. The container has one perforated side to allow the aromatic profile of the individual to escape during detection, and a lid that can be applied to prevent the aromatic cloud from escaping prior to detection. FIG. 4 shows a prototype of a collection device for collecting biological specimen. The collectors (such as cotton-tipped swab) may remain into the collection device and will not interfere with scent detection.

Each test sample is to be labeled to allow tracking and reporting.

When the test sample arrives at the lab, it remains in its airtight environment until it is ready to be screened by the animals. A track will be set up with several samples in every run. “Control Standard Sample” and a “PD Standard Sample” will be used as a quality assurance measure as negative and positive control, respectively. If the animal does not appropriately identify the PD Standard Sample as positive and the Control Standard as negative, the test will be considered invalid.

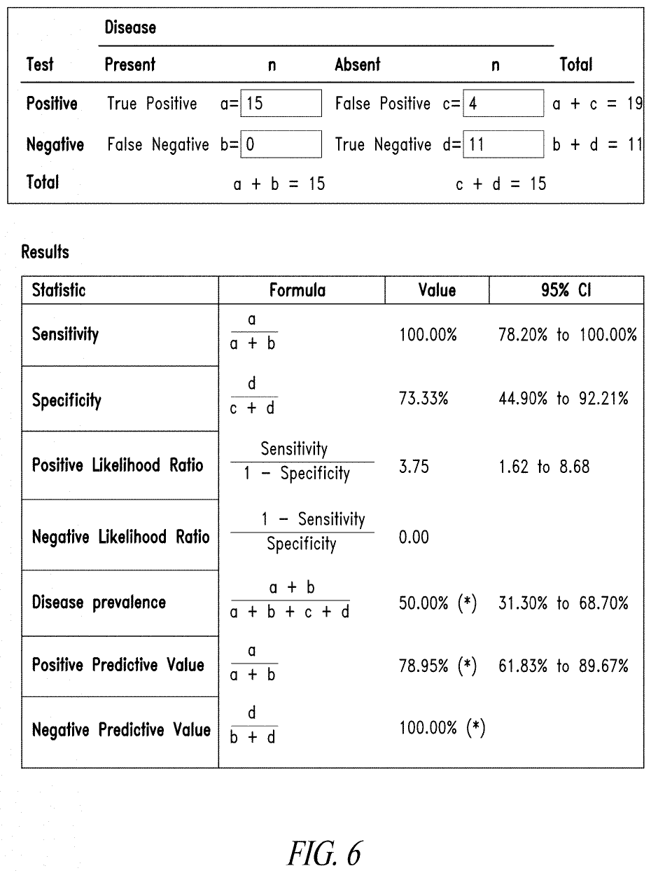

There are numerous quality control measures in place. For instance, any dog that incorrectly signals to the Standards three times in a day will be retired for the day and is not permitted to return until he/she demonstrates a capacity to discern PD Standards from Control Standards with a sensitivity and specificity meeting published standards. Statistical analyses will be performed to determine the value added by running samples in duplicate or triplicate and the ultimate decision will be based on requirements of governing bodies (e.g., Food and Drug Administration) and on the public's perception of confidence in the test. FIG. 6 shows an example of an animal trained according to the methodology described here, which in turn detected test samples with a positive predictive value of 78.95% (95% CI 61.83, 89.67%) and a negative predictive value of 100% in its ability to distinguish between samples from PD sources vs. control samples from non-PD sources.

The handler will be responsible for reading the signal of the animal.

3. Reporting and Interpretation

The sensitivity, specificity, positive predictive value, and the negative predictive value for detecting the aromatic profile, or scent, of PD will be described in the test kit and the report. For instance, given the current state of the science it will be explained that the test is not specific to idiopathic Parkinson's disease (iPD) in layman's terms. It may be possible in the future to train animals to discern between pathologically and phenotypically different types of parkinsonism.

4. Detecting Prodromal/Pre-Motor Parkinsonism

Screening guidelines will serve to identify individuals at increased risk, including those with a family history and/or non-motor symptoms common in PD. Samples that are tested positive by a trained animal in the presence of non-motor symptoms enables identification of a pre-motor cohort. As more disease-modification strategies become available, it will be increasingly important to detect the syndrome early.

### Early Detection Accompanied by Early Intervention

The animal-assisted PD screening test disclosed herein is capable of identifying individuals who may have early stage disease. One embodiment provides a method of treating the early disease following early detection, which may slow, stop and reverse the progression of parkinsonism.

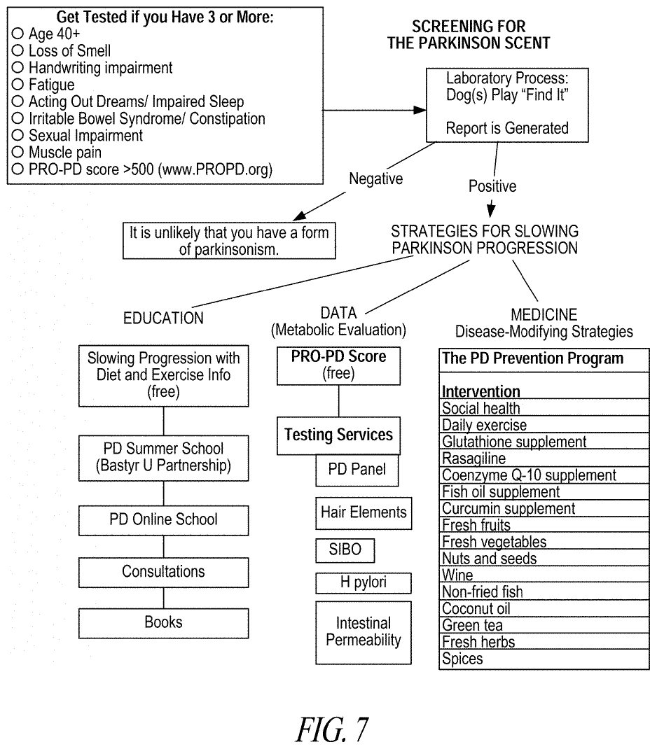

FIG. 7 summarizes an exemplary process of screening individuals in a general population to identify those at the greatest risk of parkinsonism and implementing intervention strategies for individuals that tested positive to delay or prevent the onset of parkinsonism.

For instance, it has already been established that exercise is able to slow progression of Parkinson's disease progression in individuals who have already exhibited motor symptoms. Individuals who are identified by the detection method disclosed herein, despite not having symptoms or non-motor symptoms, may implement disease-modifying strategies as a preventive measure against further accumulation of symptoms.

These disease-modifying strategies may be implemented in individuals who tested positive even though they have yet to develop motor symptoms. Certain foods and supplements are known to be associated with different rates of PD progression. See Tables 1 and 2. Supplements that reduce PD progression include, for example, glutathione (GSH) (e.g., reduced oral GSH, intranasal GSH, rectal GSH, etc.), coenzyme Q-10, dehydroepiandrosterone (DHEA), lithium, e.g., mineral salts sufficient to meet physiological requirements, and docosahexaenoic acid (DHA) have been shown to slow the progression of PD.

Likewise, pharmaceutical medications are able to diminish the rate of PD progression while others accelerate progression. Pharmaceuticals that are shown to reduce the rate of progression include, for example, MAO-B inhibitors (e.g., selegeline, rasagiline); naltrexone and levodopa.

Similar data exists for specific types of exercise.

Thus, one embodiment provides that, after the test subject is identified by the trained animals disclosed herein as having premotor parkinsonism, administering to the test subject disease-modifying therapy that is known to reduce the rate of PD progression.

TABLE 1
Multiple linear regression model of dietary intake and PD progression.
Association between dietary practices and Parkinson's disease progression

| | Mean change in | | | | Mean change in | | | |
|---|---|---|---|---|---|---|---|---|
| | PRO-PD score | | | | PRO-PD score | | | |
| Food item (serving size) | (SE)* | | P value (95% CI)* | | (SE)** | | P value (95% CI)** | |
| Fresh vegetables (½ cup) | −53.2 | (7.9) | <0.000 | (−68.7 to −37.6) | −48.9 | (8.3) | <0.000 | (−64.7 to −33.1) |
| Fresh fruit (½ cup) | −44.1 | (8.5) | <0.000 | (−60.7 to −27.5) | −40.7 | (8.6) | <0.000 | (−57.5 to −23.9) |
| Nuts (¼ cup or 2 tbsp | −38.5 | (7.5) | <0.000 | (−53.2 to −23.7) | −33.2 | (7.6) | <0.000 | (−48.1 to −18.4) |
| spread) | | | | | | | | |
| Fish (4 oz) | −37.1 | (8.9) | <0.000 | (−54.6 to −19.5) | −29.5 | (9.1) | 0.001 | (−47.3 to −11.6) |
| Olive oil (1 tsp) | −34.1 | (6.8) | <0.000 | (−47.4 to −20.8) | −31.4 | (6.8) | <0.000 | (−44.7 to −18.1) |
| Wine (6 oz) | −23.6 | (5.3) | <0.000 | (−34.1 to −13.1) | −14.6 | (5.6) | 0.009 | (−25.5 to −3.7) |
| Turkey (4 oz) | −20.2 | (18.7) | 0.281 | (−57.1 to 16.7) | −10.8 | (19.2) | 0.573 | (−48.7 to 27) |
| Coconut oil (1 tsp) | −18.6 | (5.5) | 0.001 | (−29.3 to −7.8) | −20.2 | (5.5) | <0.000 | (−31 to −9.4) |
| Fresh herbs (1 tsp) | −14.9 | (6.4) | 0.02 | (−27.4 to −2.4) | −8.9 | (6.5) | 0.169 | (−21.7 to 3.8) |
| Spices (¼ tsp) | −14.2 | (6.4) | 0.027 | (−26.7 to −1.6) | −13.4 | (6.4) | 0.037 | (−26 to −0.8) |
| Eggs (1 egg) | −9.5 | (8.2) | 0.251 | (−25.6 to 6.7) | −9.7 | (8.3) | 0.241 | (−26 to 6.5) |
| Bread (1 slice) | −7.7 | (6.8) | 0.26 | (−21.2 to 5.7) | −6.9 | (6.9) | 0.314 | (−20.4 to 6.6) |
| Beans (½ cup) | −6.3 | (8.6) | 0.466 | (−23.3 to 10.7) | −5.4 | (8.8) | 0.54 | (−22.6 to 11.8) |
| Butter (1 tsp) | −4 | (5.9) | 0.494 | (−15.6 to 7.5) | −3.8 | (6) | 0.522 | (−15.5 to 7.9) |
| Oatmeal (1 cup) | −3.2 | (6.5) | 0.624 | (−15.9 to 9.5) | −4.4 | (6.6) | 0.501 | (−17.3 to 8.5) |
| Liquor (1 oz) | −2.8 | (7.7) | 0.717 | (−17.8 to 12.3) | 3.6 | (7.7) | 0.47 | (−11.5 to 18.7) |
| Green tea (1 cup) | −2.3 | (5.7) | 0.68 | (−13.5 to 8.8) | 1.6 | (5.7) | 0.779 | (−9.6 to 12.7) |
| Juice (8 oz) | −2.3 | (5.8) | 0.687 | (−13.8 to 9.1) | −1.4 | (5.9) | 0.811 | (−12.9 to 10.1) |
| Frozen fruit (½ cup) | −1.9 | (6.1) | 0.757 | (−13.8 to 10) | −2.2 | (6.1) | 0.714 | (−14.1 to 9.7) |
| Cream (¼ cup) | −0.5 | (7.4) | 0.942 | (−15.2 to 14.1) | −0.3 | (7.4) | 0.971 | (−14.7 to 14.2) |
| Coffee (8 oz) | −0.1 | (4.4) | 0.983 | (−8.8 to 8.6) | 4.3 | (4.5) | 0.342 | (−4.5 to 13.1) |
| Soy (3 oz) | 0.4 | (7.9) | 0.962 | (−15.2 to 16) | 2.3 | (8) | 0.77 | (−13.4 to 18.1) |
| Safflower oil (1 tsp) | 0.7 | (6.9) | 0.922 | (−12.8 to 14.2) | 6.8 | (6.9) | 0.325 | (−6.8 to 20.5) |
| Beer (12 oz) | 1.1 | (7.6) | 0.88 | (−13.7 to 16) | 2 | (7.5) | 0.789 | (−12.8 to 16.8) |
| Chicken (4 oz) | 3.3 | (9.7) | 0.34 | (−15.6 to 22.3) | 13.4 | (9.8) | 0.171 | (−5.8 to 32.5) |
| Milk (1 cup) (mammalian, | 5.8 | (4.8) | 0.226 | (−3.6 to 15.2) | 5.1 | (4.8) | 0.291 | (−4.4 to 14.5) |
| for example, cow) | | | | | | | | |
| Pork (4 oz) | 6.1 | (8.6) | 0.482 | (−10.8 to 22.9) | 7 | (8.7) | 0.42 | (−10 to 24) |
| Black tea (1 cup) | 8.6 | (5.6) | 0.121 | (−2.3 to 19.5) | 8.4 | (5.6) | 0.131 | (−2.5 to 19.3) |
| Eat food from a can | 9.6 | (8.1) | 0.234 | (−6.2 to 25.4) | 6.1 | (8.1) | 0.449 | (−9.7 to 22) |
| Pasta (1 cup) | 10.1 | (9.3) | 0.28 | (−8.2 to 28.4) | 9.2 | (9.4) | 0.326 | (−9.2 to 27.6) |
| Frozen vegetables (½ cup) | 11 | (6.9) | 0.11 | (−2.5 to 24.4) | 10.3 | (6.9) | 0.137 | (−3.3 to 23.9) |
| Cheese (1 slice, 1/2 oz, 1 tbsp) | 11.7 | (6.9) | 0.091 | (−1.9 to 25.3) | 15.5 | (6.9) | 0.026 | (1.9 to 29.1) |
| Yogurt (3/4 cup) | 13.5 | (7.5) | 0.073 | (−1.3 to 28.3) | 15.2 | (7.6) | 0.046 | (0.2 to 30.1) |
| Ice cream (½ cup) | 13.8 | (7.4) | 0.064 | (−0.8 to 28.3) | 18.3 | (7.5) | 0.015 | (3.6 to 32.9) |
| Soda (12 oz) | 15.4 | (7.8) | 0.049 | (0.03 to 30.7) | 15.2 | (7.9) | 0.054 | (−0.3 to 30.6) |
| Beef (4 oz) | 16.2 | (8.3) | 0.051 | (−0.1 to 32.4) | 21.8 | (8.3) | 0.009 | (5.5 to 38.1) |
| Fried food (4 oz) | 19.5 | (8.8) | 0.027 | (2.2 to 36.8) | 23 | (8.9) | 0.009 | (5.6 to 40.4) |
| Canned vegetables (½ cup) | 19.9 | (7) | 0.005 | (6.1 to 33.6) | 18.3 | (7) | 0.009 | (4.5 to 32.1) |
| Diet soda (12 oz) | 20.7 | (6.1) | 0.001 | (8.7 to 32.8) | 23.6 | (6.1) | <0.000 | (11.6 to 35.6) |
| Canned fruit (½ cup) | 36.1 | (7.9) | <0.000 | (20.5 to 51.6) | 32 | (7.9) | <0.000 | (16.5 to 47.6) |

Predicted PD severity score, as measured by the PRO-PD, per unit increase in food intake frequency, intake measured on a 10-point scale: never, <1/month, 1/month, 2-3×/month, 1/week, 2-4×/week, 5-6×/week, 1/day, 2-4×/day, 5-6×/day.
*Adjusted for years since diagnosis, age, and gender.
**Adjusted for years since diagnosis, age, gender, and income.

TABLE 2
Logistic regression model of nutritional supplements and PD progression.
Association between dietary supplements & risk of Parkinson's disease progression.

| | | Mean change in | | | | Mean change in | | | |
|---|---|---|---|---|---|---|---|---|---|
| | | PRO-PD score | | | | PRO-PD score | | | |
| Nutritional supplement | n | (SE)* | | P value (95% CI)* | | (SE)** | | P value (95% CI)** | |
| Inosine | 13 | −181.1 | (125.6) | 0.15 | (−427.5 to 65.3) | −107.1 | (122.9) | 0.384 | (−348.4 to 134.2) |
| Glutathione, oral | 43 | −126.1 | (69) | 0.068 | (−261.6 to 9.3) | −126.7 | (70) | 0.07 | (−263.9 to 10.5) |
| DHEA | 47 | −87.6 | (70.8) | 0.216 | (−226.6 to 51.4) | −72.2 | (70.9) | 0.309 | (−211.3 to 67) |
| Lithium, low dose | 21 | −84.9 | (100.2) | 0.397 | (−281.6 to 111.8) | −118.9 | (100.4) | 0.237 | (−315.9 to 78.1) |
| Low-dose naltrexone | 14 | −76.1 | (120.9) | 0.529 | (−313.4 to 161.2) | −87.8 | (118) | 0.457 | (−319.3 to 143.8) |
| CoQ10 | 286 | −70.4 | (31.5) | 0.026 | (−132.2 to −8.6) | −46.6 | (31.6) | 0.141 | (−108.7 to 15.4) |
| Fish oil | 376 | −69.5 | (29.5) | 0.019 | (−127.4 to −11.6) | −57.7 | (29.6) | 0.052 | (−115.7 to 0.4) |
| Quercetin | 21 | −50.7 | (105.9) | 0.632 | (−258.5 to 157.1) | −60.5 | (106.4) | 0.569 | (−269.3 to 148.2) |
| Turmeric/curcumin | 197 | −47.3 | (35.6) | 0.186 | (−117.3 to 22.8) | −49.5 | (35.9) | 0.168 | (−120 to 20.9) |
| Gingko biloba | 30 | −47.2 | (83.2) | 0.57 | (−210.5 to 116) | −61.1 | (81.2) | 0452 | (−220.5 to 98.2) |
| Coconut oil | 190 | −35.8 | (36.4) | 0.324 | (−107.2 to 35.5) | −52.7 | (36.4) | 0.147 | (−124.1 to 18.6) |
| Resveratrol | 43 | −28.5 | (70.7) | 0.687 | (−167.3 to 110.3) | −18.7 | (72.7) | 0.797 | (−161.4 to 124) |
| Vitamin D | 623 | −26.1 | (29) | 0.368 | (−83 to 30.8) | −3.6 | (29.2) | 0.902 | (−60.9 to 53.7) |
| Alpha-lipoic acid | 79 | −19.1 | (53.4) | 0.72 | (−123.9 to 85.7) | 0.05 | (54.4) | 0.999 | (−106.7 to 106.7) |
| 5-Methyltetrahydrofolate (5- | 27 | −17.1 | (91.4) | 0.852 | (−196.4 to 162.2) | −25.1 | (95.6) | 0.793 | (−212.7 to 162.5) |
| MTHF) | | | | | | | | | |
| Probiotics | 249 | −12.3 | (32.7) | 0.708 | (−76.5 to 52) | −12.4 | (32.9) | 0.706 | (−77 to 52) |
| NADH | 14 | −9.7 | (120.8) | 0.936 | (−246.7 to 227.3) | −25.2 | (122.6) | 0.837 | (−265.7 to 215.4) |
| Multivitamin/mineral | 342 | −7.8 | (30.2) | 0.795 | (−67.1 to 51.4) | 9.9 | (30.3) | 0.744 | (−49.6 to 69.5) |
| Calcium | 324 | −6.2 | (32.2) | 0.847 | (−69.4 to 57) | 12.5 | (32.6) | 0.701 | (−51.4 to 76.4) |

Predicted PD severity score, as measured by the PRO-PD, based on the positive report of consistently using of supplements over the previous 6 months.
*Adjusted for years since diagnosis, age, and gender.
**Adjusted for years since diagnosis, age, gender, and income.

### Examples

### Canine Capacity to Distinguish PD Standards from Control Standards

A dog of Lagotto romagnolo breed trained according to the methodology described here has demonstrated a positive predictive value of 78.95% (95% CI 61.83, 89.67%) and a negative predictive value of 100% in its ability to distinguish between samples from individuals with PD from samples from non-PD, healthy controls after ten months of training.

FIG. 5 shows 53 consecutive exposures run on a track using only cotton-tipped swabs collected from the scraping of the ear canal. For this test, all 15 controls were collected from individuals who met criteria of non-PD sources as disclosed herein, i.e., they have denied non-motor symptoms common in PD, e.g., impaired sleep, fatigue, restless leg syndrome, constipation, anosmia, etc. All were from middle-aged adults.

All 15 PD samples were from individuals who met the criteria for PD sources, e.g., they have exhibited one or more symptoms including unilateral onset, levodopa improves tremor by at least 70%, rigidity, etc. All 15 PD Samples also had evidence of reduced dopamine transporter activity with SPECT imaging, i.e., DaTscan positive.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

U.S. provisional patent application Ser. No. 62/446,433 filed Jan. 14, 2017 is incorporated herein by reference, in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for training an animal for detecting and recognizing Parkinson-distinctive scent, the method comprising:
providing a training sample including excrements derived from one or more PD sources, each PD source being a person having shown at least one clinical marker for PD;
providing a control sample including excrements derived from one or more non-PD sources showing no clinical marker for PD;
allowing the animal to associate the training sample with PD by exposing the animal to a training sample in combination with a reward; and
training the animal to discriminate between the training sample and the control sample by allowing the animal to smell the training sample and the control sample in the absence of any reward and rewarding the animal only when the animal correctly identifies the training sample.

2. The method of claim 1 wherein the at least one clinical marker is levodopa-responsiveness, bradykinesia, unilateral onset, progressive disorder, muscular rigidity, resting tremor, or postural instability.

3. The method of claim 1 wherein the non-PD sources have no pre-motor symptoms.

4. The method of claim 3 wherein the pre-motor symptoms include loss of smell, constipation, restless leg syndrome, depression, rapid eye movement sleep behavior disorder (RBD), fatigue, impaired sleep, impaired balance, sexual impairment, urinary dysfunction, impaired cognition, or a combination thereof.

5. The method of claim 1 wherein the training sample includes one or more excrements selected from the group consisting of ear wax, skin cells, organisms and their metabolites from the dermal microbiome, stool, sebum, urine, and saliva.

6. The method of claim 1 wherein the control sample includes one or more excrements selected from the group consisting of ear wax, skin cells, organisms and their metabolites from the dermal microbiome, stool, sebum, urine, and saliva.

7. The method of claim 1 further comprising interpreting the animal's signal of identification to determine presence or absence of Parkinson-distinctive scent.

8. The method of claim 1 wherein the animal is a canine or a swine.

9. The method of claim 1 wherein the one or more PD sources are patients having positive diagnosis of idiopathic Parkinson's disease (iPD), Parkinson's disease, Alzheimer's disease (AD), Lewy body dementia (LBD), multiple system atrophy (MSA), corticobasal degeneration (CBD), progressive supranuclear palsy (PSP), or a combination thereof.

10. A Parkinson (PD)-scented training sample comprising: one or more excrements derived from one or more PD sources, each PD source being a person having shown at least one clinical marker for PD.

11. The PD-scented training samples of claim 10 wherein the one or more excrements are selected from the group consisting of ear wax, skin cells, organisms and their metabolites from the dermal microbiome, stool, urine, and saliva.

12. The PD-scented training sample of claim 11 wherein the one or more excrements collected from different PD sources are of a same type.

13. The PD-scented training sample of claim 12 wherein the one or more excrements collected from different PD sources are all ear wax.

14. A method of preventing or delaying the onset of motor symptoms of PD in a test subject, the method comprising:
allowing an animal trained by the method according to claim 1 to smell the test subject or a test sample containing excrement extracted from the test subject; and
interpreting a response given by the animal to determine presence or absence of Parkinson-distinctive scent.
screening the test subject for Parkinson-distinctive scent for non-motor symptoms or genetic testing; and
administering a disease-modifying therapy to the test subject tested positive for Parkinson-distinctive scent.

15. The method of claim 14 wherein the disease-modifying therapy includes one or more supplements selected from the group consisting of glutathione (GSH), coenzyme Q-10, dehydroepiandrosterone (DHEA), lithium, and docosahexaenoic acid (DHA).

16. The method of claim 14 wherein the disease-modifying therapy includes MAO-B inhibitors, naltrexone, levodopa or a combination thereof.

17. A method of training an animal to respond to Parkinson-distinctive scent, the method comprising exposing the animal to a training sample of claim 10 in combination with a reward, wherein the training sample emits Parkinson-distinctive scent.

18. The method of claim 17 further comprising reinforcing a positive association of Parkinson-distinctive scent and reward by further rewarding the animal upon recognition of Parkinson-distinctive scent.
